(19) United States
(12) Patent Application Publication
(10) Pub. No.: US 2021/0241037 A1
(43) Pub. Date: Aug. 5, 2021

(54) DATA PROCESSING APPARATUS AND METHOD

(71) Applicant: CANON MEDICAL SYSTEMS CORPORATION, Otawara-shi (JP)

(72) Inventor: Aneta LISOWSKA, Edinburgh (GB)

(73) Assignee: CANON MEDICAL SYSTEMS CORPORATION, Otawara-shi (JP)

(21) Appl. No.: 16/919,329

(22) Filed: Jul. 2, 2020

Related U.S. Application Data
(60) Provisional application No. 62/967,963, filed on Jan. 30, 2020.

(51) Int. Cl. G06K 9/62; G16H 30/40; G06N 20/20

(52) U.S. Cl. CPC G06K 9/6257; G06K 9/6259; G06N 20/20; G16H 30/40; G06K 9/6228

(57) ABSTRACT

A data processing apparatus for training models on data, comprises processing circuitry configured to:
train a first model on a plurality of labelled data sets;
apply the first trained model to a plurality of non-labelled data sets to obtain first pseudo-labels;
train a second model using at least the labelled data sets, the non-labelled data sets and the first pseudo-labels;
apply the second trained model to non-labelled data sets to obtain second pseudo-labels; and
train a third model based on at least the labelled data sets, non-labelled data sets and the second pseudo-labels.

### FIELD

Embodiments described herein relate generally to a method and apparatus for processing data, for example for training a machine learning model and/or labelling data sets.

### BACKGROUND

It is known to train machine learning algorithms to process data, for example medical data.

Training of machine learning models can be performed using either supervised or unsupervised techniques, or a mixture of supervised and unsupervised techniques.

Supervised machine learning techniques require large amounts of annotated training data to attain good performance. However, annotated data is difficult and expensive to obtain, especially in the medical domain where only domain experts, whose time is scarce, can provide reliable labels. Active learning (AL) aims to ease the data collection process by automatically deciding which instances an expert should annotate in order to train a model as quickly and effectively as possible. Nevertheless, the unlabelled datasets do not actively contribute to model training, the amount of data, and the annotation requirements are potentially still large

Features in one aspect or embodiment may be combined with features in any other aspect or embodiment in any appropriate combination. For example, apparatus features may be provided as method features and vice versa.

### BRIEF DESCRIPTION OF THE DRAWINGS

Embodiments are now described, by way of non-limiting example, and are illustrated in the following figures, in which:

FIG. 1 is a schematic illustration of an apparatus in accordance with an embodiment;

FIG. 2 is a schematic illustration of certain stages of a process according to an embodiment that includes training of a master model and a student model as part of a multi-stage model training process;

FIG. 3 is a schematic illustration, in more detail, of certain stages of a process according to an embodiment that includes training of a master model and student models as part of a multi-stage model training process;

FIG. 4 is a schematic illustration in overview of a process according to an embodiment, which uses processes as described in relation to FIGS. 3 and 4, and which includes training a master model and a plurality of student models;

FIG. 5 is a plot of accuracy of segmentation of lung, heart, oesophagus, and spinal cord from certain test data sets versus number of models used in a series of pseudo-labelling and training processes, achieved using an embodiment;

FIG. 6 includes scan images of heart, oesophagus, and spinal cord, and corresponding segmentations obtained according to an embodiment using a succession of models; and

FIG. 7 includes scan images of heart, oesophagus, and spinal cord together with corresponding ground truth, uncertainty, and error measures.

### DETAILED DESCRIPTION

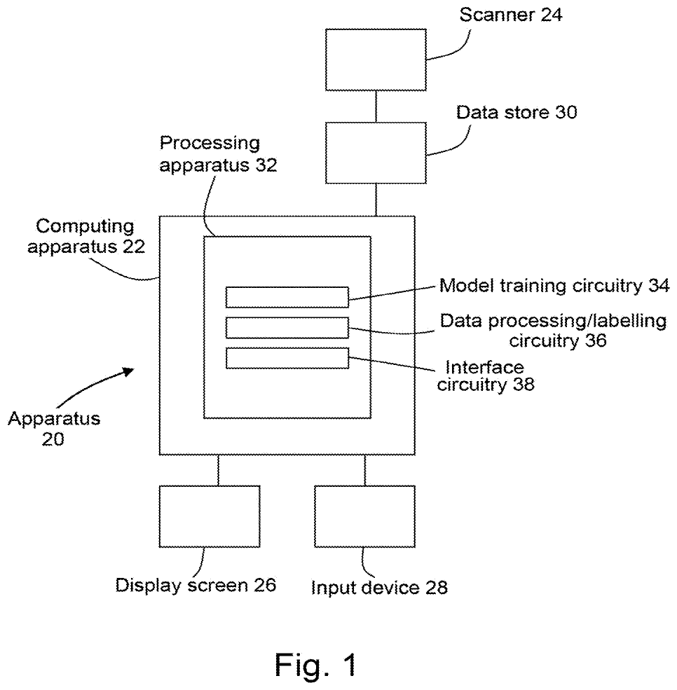

A data processing apparatus 20 according to an embodiment is illustrated schematically in FIG. 1. In the present embodiment, the data processing apparatus 20 is configured to process medical imaging data. In other embodiments, the data processing apparatus 20 may be configured to process any appropriate data, for example imaging data, text data, structured data, for example graph data such as an ontology tree, or a combination of heterogeneous data.

The data processing apparatus 20 comprises a computing apparatus 22, which in this case is a personal computer (PC) or workstation. The computing apparatus 22 is connected to a display screen 26 or other display device, and an input device or devices 28, such as a computer keyboard and mouse.

The computing apparatus 22 is configured to obtain image data sets from a data store 30. The image data sets have been generated by processing data acquired by a scanner 24 and stored in the data store 30.

The scanner 24 is configured to generate medical imaging data, which may comprise two-, three- or four-dimensional data in any imaging modality. For example, the scanner 24 may comprise a magnetic resonance (MR or MRI) scanner, CT (computed tomography) scanner, cone-beam CT scanner, X-ray scanner, ultrasound scanner, PET (positron emission tomography) scanner or SPECT (single photon emission computed tomography) scanner.

The computing apparatus 22 may receive medical image data from one or more further data stores (not shown) instead of or in addition to data store 30. For example, the computing apparatus 22 may receive medical image data from one or more remote data stores (not shown) which may form part of a Picture Archiving and Communication System (PACS) or other information system.

Computing apparatus 22 provides a processing resource for automatically or semi-automatically processing medical image data. Computing apparatus 22 comprises a processing apparatus 32. The processing apparatus 32 comprises model training circuitry 34 configured to train one or more models; data processing/labelling circuitry 36 configured to apply trained model(s) to obtain outputs and/or to obtain labels, for example to obtain labels, pseudo-labels, segmentations or other processing outcomes, for example for output to a user or for providing to the model training circuitry 34 for further model training processes; and interface circuitry 38 configured to obtain user or other inputs and/or to output results of the data processing.

In the present embodiment, the circuitries 34, 36, 38 are each implemented in computing apparatus 22 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

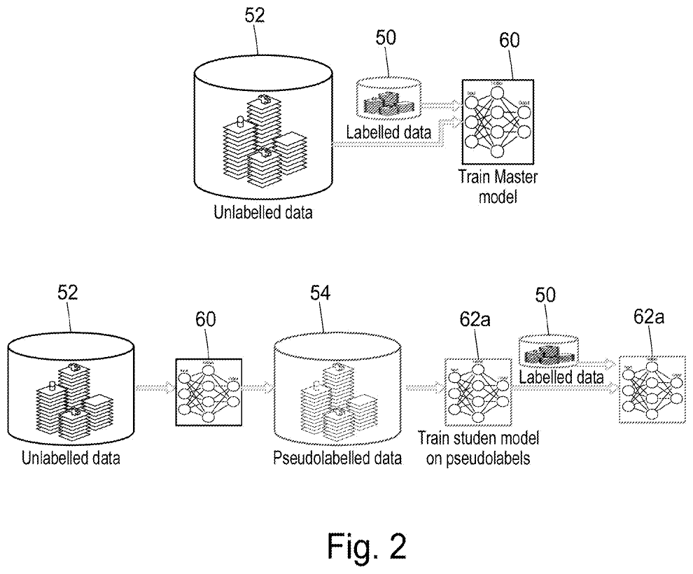

The computing apparatus 22 also includes a hard drive and other components of a PC including RAM, ROM, a data bus, an operating system including various device drivers, and hardware devices including a graphics card. Such components are not shown in FIG. 2 for clarity.

The data processing apparatus 20 of FIG. 1 is configured to perform methods as illustrated and/or described in the following.

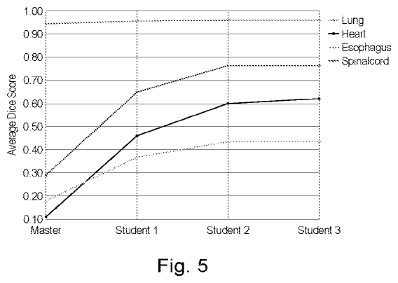

It is a feature of embodiments that at least three models are used in a training process that involves both labelled and unlabelled data. The models can be referred to as a master model and subsequent student models of a series. Processes involved in the training of the master model and student models are described in relation to FIGS. 2, 3 and 4. The effect of the number of models used on accuracy of labelling according to some embodiments is then considered with reference to FIGS. 5 to 7.

The model training circuitry 34 uses both sets of labelled data 50 and sets of unlabelled data 52 in training the master model 60 and student models 62a . . . n. The embodiment of FIG. 1 is able to use the labelled data 50 and unlabelled data 52 in a semi-supervised active learning process.

As illustrated schematically in FIG. 2, in the semi-supervised active learning process the models can ultimately be trained both on the labelled data 50 and the unlabelled data 52 for example based on loss consisting of two parts: 1) standard pathology classification loss in relation to the labelled data and 2) uncertainty minimisation loss in relation to the labelled and unlabelled data.

Furthermore, as also illustrated schematically in FIG. 2, the master model can use the unlabelled data 52 to predict labels for at least some of the unlabelled data. The predicted labels can be referred to as pseudo-labels and the combination of the unlabelled data with associated pseudo-labels referred to us pseudo-labelled data 64. Pseudo-labels can be labels generated in any way other than by a human expert, for example generated automatically by a model. As shown schematically in FIG. 2, a first student model 62a can then be trained using the pseudo-labelled data 54 (e.g. the combination of the unlabelled data 52 and its associated pseudo-labels) and the student model 62a can subsequently be fine tuned using, in addition, the labelled data 50.

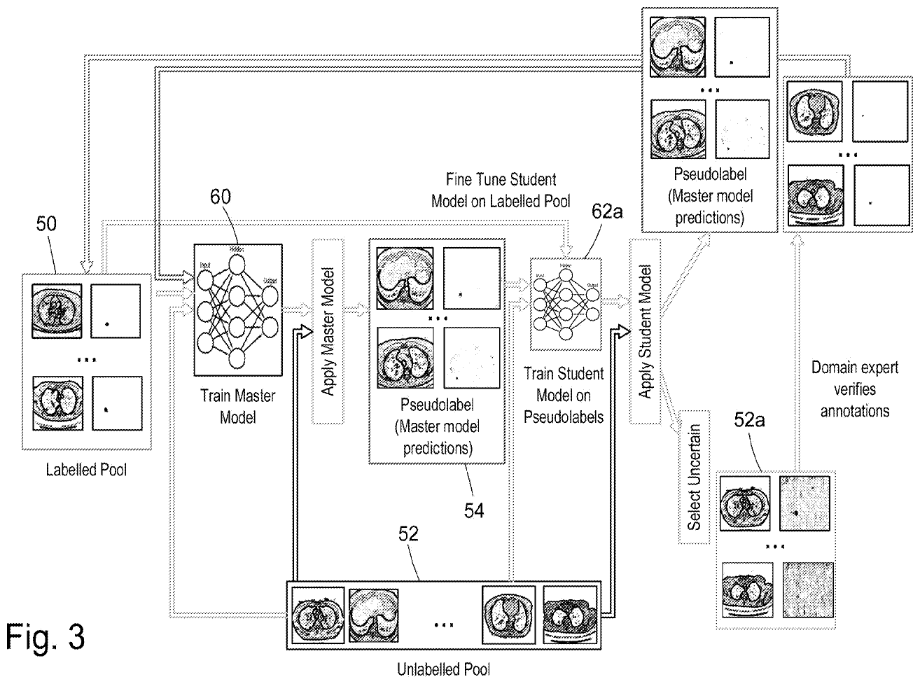

Before going on to consider further use of series of successively more refined student models according to embodiments, training processes for the master model 60 and student model 62a are considered in more detail in relation to FIG. 3.

As already noted, the training process is performed by the model training circuitry 34 using a combination of labelled datasets 50 and unlabelled datasets 52. The labelled datasets 50 may be obtained in any suitable fashion. In the embodiment of FIG. 3 the labelled datasets 50 are obtained by an expert (for example a radiologist and/or expert in particular anatomical features, conditions or pathologies under consideration) annotating a small subset of the available relevant datasets.

The labels of the labelled dataset can be of any type suitable for a learning and/or processing task under consideration. For instance if the models are be used for segmentation purposes, the labels may identify which pixels or voxels, or regions of pixels or voxels, correspond to an anatomical feature and/or pathology of interest. Any other suitable labels may be used, for example labels indicating or more properties of subject, for instance a patient, such as presence, absence or severity of a pathology or other condition, age, sex, weight, of conditions, and/or labels indicating one or more properties of an imaging or other procedure performed on the subject. As mentioned further below, embodiments are not limited to using imaging data, and other types of labelled and unlabelled datasets are used, including for example text data.

Returning to the details of FIG. 3, at a first stage the model training circuitry 34 trains a master model 60 using the labelled datasets 50. In the embodiment of FIG. 3 the master model 60 is a neural network trained. Certain training techniques used in the embodiment of FIG. 3 are discussed further below. In alternative embodiments any suitable models for example any suitable machine learning or other models, for instance a random forest model, and any suitable training techniques may be used.

Once the master model 60 has been trained using the labelled datasets 50, the master model 60 is applied to the unlabelled datasets 52 by the data processing/labelling circuitry 36 to generate pseudo-labels for the unlabelled datasets. In the present embodiment the labels and pseudo-labels are used for segmentation of the imaging data represent segmentations (for example, which pixels or voxels, or regions of pixels or voxels, correspond to an anatomical feature and/or pathology of interest) and the pseudo-labels generated by the master model 60 represent the predictions, for each unlabelled dataset, as to whether pixels or voxels of the unlabelled dataset correspond to an anatomical feature of interest or not.

A first student model 62a is then trained using the pseudo-labelled data set 54 (e.g. the combination of the unlabelled datasets 52 and the associated pseudo-labels generated by the master model 60). In the present embodiment the student models 62a . . . n are of the same type as the master model 60 and are neural networks. In alternative embodiments, at least some or all of the student models 62a . . . n may be of different types and/or have different properties to the master model.

Next, the training of the student model 62a is fine-tuned using the labelled datasets 50. The combination of the training using the labelled datasets 50 and the training (e.g. fine tuning) using the unlabelled datasets may be performed in any suitable fashion, for example with the initial training using the unlabelled datasets 52 being followed by fine tuning using the labelled datasets 50, or with the training using labelled datasets 50 and unlabelled datasets 52 being performed simultaneously or in other combined fashion.

At the next stage the trained student model 62a is applied by the processing circuitry 36 to the unlabelled datasets 52, to select at least some of the unlabelled datasets 52a for which labelling by an expert may be desirable, and/or to provide pseud-olabels for at least some of the unlabelled datasets. The providing of pseudolabels for at least some of the unlabelled datasets 52 may comprise, for example, modifying or replacing pseudo-labels provided by the master model for those unlabelled datasets 52.

The selection of the unlabelled datasets 52a for which labelling by an expert may be desirable may be performed based on any suitable criteria. For example, unlabelled datasets for which the pseudo-labelling seems to be particularly low quality (e.g. below a threshold measure of quality) or uncertain may be selected. Alternatively, unlabelled data sets may be selected dependent on how representative of, and/or similar to, other of the unlabeled data sets they are. Any other suitable sampling strategies may be used to select the unlabelled data sets.

Once the selected unlabelled datasets have been labelled by the expert, for example using interface circuitry 38 or in any other suitable manner, they then form part of an updated set of labelled datasets 50. Thus, the number of sets of labelled data 50 increases. The number of set of unlabeled data 52 correspondingly decreases.

In some embodiments, at least some of the pseudo-labelled datasets (e.g. at least some of the unlabelled datasets 52 that are pseudo-labelled by the student model 62a) are also included in the modified labelled dataset 50.

The processes are then iterated, with the first student model 62a effectively becoming a new master model 60 in the schematic diagram of FIG. 3. The first student model 62a (which we can consider as a new master model) is then trained on the updated labelled data set 50 before being applied, and a new student model 62b is then trained and applied, in line with the processes described above, but with the new student model 62b place of the initial student model 62a. Further unlabeled data sets are then labelled by an expert and/or pseudo-labelled by the student model 62b and the sets of labelled and unlabelled data are further updated, and the training, applying and updating processes may be repeated with a new student model 62c or the iterative process may be ended.

Once the iterative process is ended then the last student model that has been trained may be considered to be a final model.

Before considering the iterative nature of the procedure in more detail, it has already been noted that any suitable training process of the models may be used. It is a feature of the embodiment of FIGS. 2 and 3 that the updated master model (corresponding to e.g. first, second or subsequent student models in subsequent iterations) can be trained using loss consisting of two parts: 1) pathology classification/regression loss (for example, binary cross entropy, or mean squared error) based on the labelled data sets and pseudo-labelled data sets (e.g. the combination of unlabelled data sets and associated pseudo-labels generated as part of the iterative procedure) and 2) uncertainty minimisation loss (for example, minimising variance) with respect to the labelled and unlabelled datasets 50, 52. This approach can be an effective way to use both labelled and unlabelled data sets in the training process.

The uncertainty minimisation loss component of the training process with respect to the labelled and unlabelled datasets 50, 52 can be implemented in similar manner to that described in Jean et al (“Semi-supervised Deep Kernel Learning: Regression with Unlabeled Data by Minimizing Predictive Variance”, 32^{nd} Conference on Neural Information Processing Systems (NeurIPS2018)) in which an an unsupervised loss term that minimizes the predictive variance for unlabelled data can be used together supervised loss term(s). An understanding that uncertainty of a model can be estimated by incorporating a dropout layer activated at inference time, with the variance between the prediction of the model reflecting the model uncertainty, see for example Yarin Gal et al, “Dropout as a Bayesian Approximation: Representing Model Uncertainty in Deep Learning”, Proceedings of the 33^{rd} International Conference on Machine Learning, PMLR 48, 1050-1059, 2016.

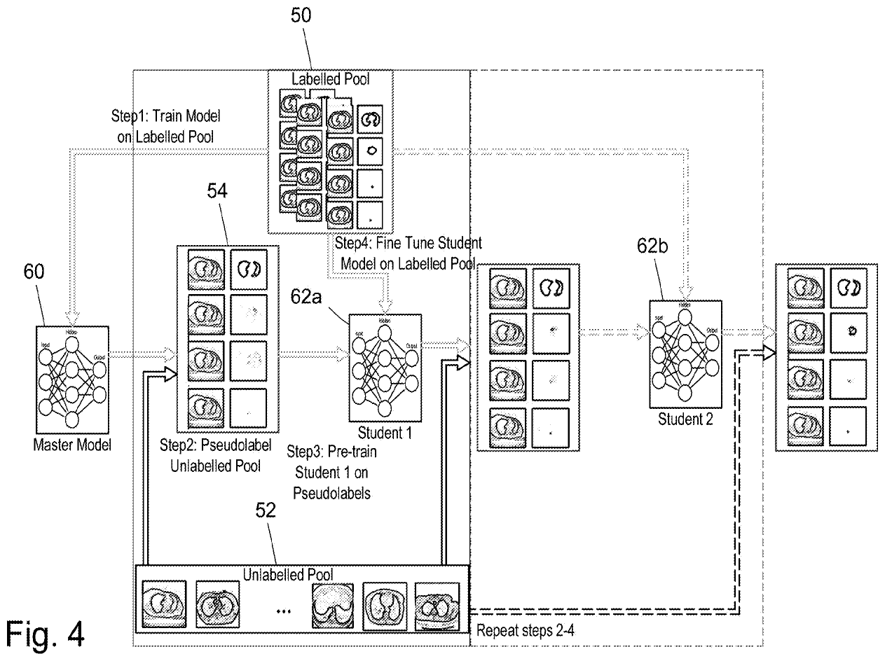

Returning to the iterative nature of the procedure, as outlined above, FIG. 4 is a schematic illustration of operation of an embodiment similar to that of FIG. 3. The steps of training a model (the master model initially) on the sets of labelled data 50, followed by pseudo-labelling the sets of unlabelled data 52 using the trained model, followed by training based on the pseudo-labelled data, followed by fine tuning the student model are labelled as steps 1 to 4 on the figure, with the steps then being repeated with the master model be replaced by the trained and fine-tuned student model, and a further student model (e.g. student model 2) replacing the student model (e.g. student model 1) in the next iteration.

As mentioned above in relation to FIG. 3, the training, applying and updating steps may then be repeated, iteratively, with new student model(s) or the iterative process may be ended. Once the iterative process is ended then the last student model that has been trained may be considered to be a final model.

The final model can then be stored and/or used for subsequent classification or other task by applying the trained model to one or more datasets, for example medical imaging datasets, to obtain a desired result. The trained model may be applied to imaging or other datasets to obtain an output representing one or more of a classification, a segmentation, and/or an identification of an anatomical feature or pathology.

Any suitable types of medical imaging data may be used as data sets in the training process or may be the subject of application of the final model following the training. For example, the data sets may comprise one or more of magnetic resonance (MR) data sets, computed tomography (CT) data sets, X-ray data sets, ultrasound data sets, positron emission tomography (PET) data sets, single photon emission computed tomography (SPECT) data sets according to certain embodiments. In some embodiments the data may comprise text data or any other suitable type of data as well as or instead of imaging data. For instance, in some embodiments the data comprises patient record datasets or other medical records.

It is has been found for at least some embodiments that the number of iterations of the procedure, for example the number of student models and associated iterations that are used, can have an effect on the accuracy of training and/or the accuracy of output of the resulting final model.

FIG. 5 is a plot of average Dice score obtained for a trained model of the embodiment of FIG. 3 based on a comparison between segmentations of various anatomical features (lung, heart, oesophagus, spinal cord) obtained for imaging datasets and the corresponding ground truth segmentations for those data sets determined by an expert. It can be seen that the accuracy of the segmentations obtained by the final model increases with the number of iterations (i.e. the number of student models) used in the training process.

In practice, according to certain embodiments there can be a trade-off between the number of iterations (i.e. the number of models) to obtain increased accuracy and the time and computing resources needed to train increasing number of models. The number of models/iterations chosen may depend on the nature of the classification, segmentation or other task the models are to be used for, the nature and amount of training data, and the available computing resources. In some embodiments, between 3 and 20 successive models are used in the iterative training process, for example between 3 and 16 models, or 3 and 10 models. For example, in one embodiment relating to histology classification 5 successive models were used. In another embodiment, relating to heart segmentation 16 successive models were used. The number of models may depend on the application and/or the quality and amount of data, and may in some embodiments be selected by a user.

In some embodiments, instead of having a fixed number of iterations, a termination condition can be applied to determine when to terminate the training procedure. The training procedure may continue, with increasing numbers of iterations/models until the termination condition is achieved. The termination condition in some embodiments may comprises one or more of achievement of a desired output accuracy, a predicted or desired performance, an amount of labelled data, a desired proportion of number of labelled data sets to number of unlabeled data sets, a number of iterations reaching a threshold value, or there being no (or less than a threshold amount of) improvement in comparison to that achieved by previous iteration(s).

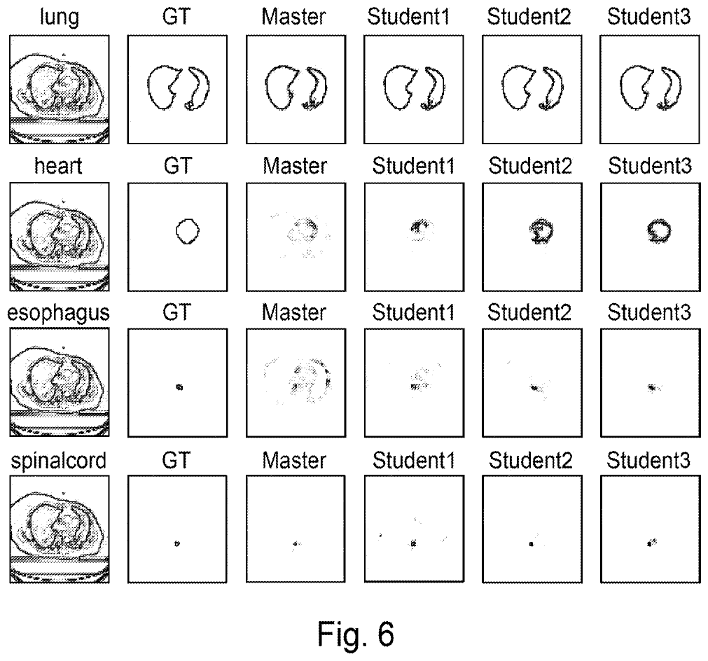

FIG. 6 shows scan images of the heart, oesophagus, and spinal cord used to obtain the results of the plot of FIG. 5, and the corresponding segmentations obtained by the final model when using a trained master model only, or a master model and one, two or three student models, in the training process of FIGS. 3 and 4 to obtain the trained final model. The ground truth segmentation is also shown.

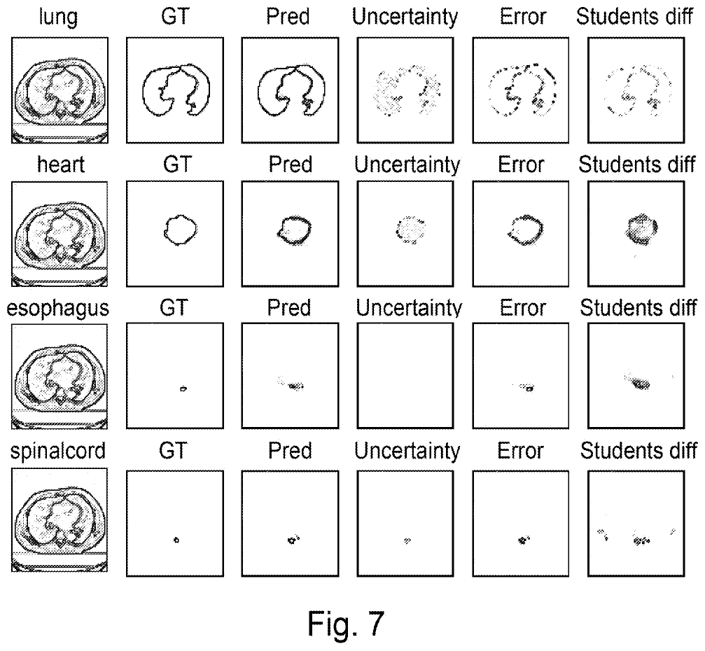

FIG. 7 shows scan images of the heart, oesophagus, and spinal cord used in another example together with corresponding ground truth, predictions obtained using models trained according to embodiments, uncertainty measures, and error measures obtained using models trained according to embodiments. It is a feature of embodiments, based upon iterative training of a succession of student models, that the difference between predictions of the models in the training chain can provide an uncertainty measure which correlates more strongly with the model error that the uncertainty of any one model. This enables use of uncertainty minimisation loss alongside the supervised loss even in an active learning set up.

Certain embodiments provide a data processing apparatus for training models on data, comprising processing circuitry configured to:
train a model on a labelled sub-set of the data;
apply the trained model to the data to select and automatically label a further sub-set of the data;
train a further model using at least the labelled sub-set and the further automatically labelled sub-set;
use the further model to select further sub-set(s) of the data to be labelled, and/or to select at least some of the automatically labelled sub-set or the labelled sub-set for verification or modification of labels.

The processing circuitry may use the further model to label automatically said further sub-set(s) of the data.

The processing circuitry may be configured to provide an output identifying said further sub-set(s) of data for manual labelling by a user and/or identifying at least some of the automatically labelled sub-set or the labelled sub-set for verification or modification of labels by a user.

The processing circuitry may be configured to provide the further sub-set(s) of labelled data and/or modified sub-set(s) of labelled data to the model, to the further model or to an additional further model for use in training.

The processing circuitry may be configured to perform a series of training and labelling processes in respect of the data, for example thereby increasing the amount of the data that is labelled and/or increasing an accuracy of the labelling and/or increasing an accuracy of model output.

The series of training and labelling processes may be performed using a series of additional further models.

The series of labelling processes may comprise automatically labelling data and/or labelling based on user input.

The model, the further model and/or the at least one additional further model may have substantially the same structure, optionally may be substantially the same. The model, the further model and/or the at least one additional further model may comprise have different starting set-ups, for example different starting weights, for example substantially randomised starting weights and/or a substantially randomised initial layer.

The series of additional further models may comprise at least one additional further model, optionally at least 5 additional further models, optionally at least 10 additional further models, optionally at least 100 additional further models.

The series of labelling and training responses may be terminated in response to an output accuracy, a predicted performance, an amount of labelled data, or a number of iterations reaching a threshold value.

The processing circuitry may be configured to repeat the training and application of the model and/or further model thereby to refine the model and/or such that increasing amounts of labelled data are used in training of the model. The model may be replaced by the further model in the repeating of the training and application, and the further model may be replaced by at least one additional further model.

The processing circuitry may be configured to apply the trained further model to a data set to obtain an output.

The processing circuitry may be configured to apply the trained additional further model to a data set to obtain an output.

The data set may comprise a medical imaging data set and the output may comprise or represent a classification and/or a segmentation and/or an identification of an anatomical feature or pathology.

The data set may comprise an imaging data set, for example a set of pixels or voxels. The output may comprise or represent a classification and/or a segmentation and/or an identification of at least one feature of an image. The output may comprise a set of labels.

The data set may comprise text data. The output may comprise diagnosis data and/or suggested treatment data and/or supplemental data to supplement the data set and/or inferred or extrapolated data, and/or correction data to correct at least part of the data set.

The training may be based on loss.

At least some of the training may be based on a combination of classification and uncertainty minimisation.

At least some of the training may be based on determination of classification loss value(s) for the labelled sub-set and determination of uncertainty minimisation loss value(s) for the unlabelled sub-set and/or the labelled sub-set alone or in combination.

The uncertainty minimisation may comprise estimating uncertainty using a dropout layer of the model and/or further model and/or additional further model(s).

The training and/or labelling may comprise or forms part of an active learning process.

The training of the model and/or the further model may comprise using different weightings in respect of labelled and unlabelled data.

The training of the model and/or the further model may be performed also using an unlabelled sub-set of the data.

The training of the model and/or further model and/or additional further model(s) may comprise or form parts of a machine learning method, e.g. a deep learning method. The training may comprise mimimizing loss, for example using one of uncertainty minimization, self-reconstruction, normalized cut. The training may comprise mimimizing loss, for example including applying different weights for labelled and unlabelled data. The processing circuity may be configured to perform training and/or labelling and/or applying processes in a distributed manner, for example with models and/or annotators/labellers distributed across different locations. Each of the model and/or the further model and/or the at least one additional further model may comprise an ensemble of trained models.

The data may comprise medical imaging data or text data.

The medical imaging data may comprise sets of pixels or voxels.

The data may comprise a plurality of data sets, and the sub-set(s) of data comprise a selected plurality of the data sets.

The data may comprise at least one magnetic resonance (MR) data, computed tomography (CT) data, X-ray data, ultrasound data, positron emission tomography (PET) data, single photon emission computed tomography (SPECT) data, or patient record data.

Labels of the labelled sub-set(s) of data comprise or represent a classification and/or a segmentation and/or an identification of an anatomical feature or pathology.

Certain embodiments provide a method of training models on data, comprising:
training a model on a labelled sub-set of the data;
applying the trained model to the data to select and automatically label a further sub-set of the data;
training a further model using at least the labelled sub-set and the further automatically labelled sub-set;
using the further model to select further sub-set(s) of the data to be labelled, and/or to select at least some of the automatically labelled sub-set or the labelled sub-set for verification or modification of labels.

Certain embodiments provide Certain embodiments provide a method of a training a model on a set of data comprising:
training the model on a labelled sub-set of the data;
applying the trained model to the set of data to select and automatically label a further sub-set of the data;
training a further model using at least the labelled sub-set and the further automatically labelled sub-set;
using an output of the further model to select further sub-set(s) of the data to be labelled, and/or labelling automatically further sub-set(s) of the data using the output of the further model;
providing the further sub-set(s) of labelled data to the model and further training the model using the further sub-set(s) of labelled data.

Certain embodiments provide a method for semi-supervised medical data annotation and training comprising using machine learning models, a pool of labelled data and a pool of unlabelled data.

Initial small labelled samples may be annotated/labelled by clinical expert/s or expert system (legacy algorithm/s).

A master model (either initialised randomly or from pretrained model) may be trained in a semi-supervised fashion using both labelled and unlabelled data pool.

The master model may annotate/label the unlabelled data after training, either for purpose of sample selection or for use in further training.

A student model (either initialised randomly or from pretrained model) may be trained on pseudo-labels generated by master model, either in fully supervised fashion or as master model is semi-supervised way.

The student model may be fine tuned on the labelled data (some part of the network may be frozen but not necessarily).

The student model may annotate/label the unlabelled data after training, either for purpose of sample selection or for use in further training.

A subset of the unlabelled data may be selected for expert/s and/or external system annotation/labelling or verification. The selection can be done automatically using model outputs (for example any combination of uncertainty, representativeness, accuracy, randomly sampling) or manually by human expert.

Reannotated/relabelled or verified samples may be added to the labelled pool.

The student model may become a master in next learning iteration and new student model may be created.

The master model in the next active learning iteration may be trained on labelled samples and pseudo-labelled samples and/or unlabelled samples in semi-supervised fashion. Where the contribution of each data pool may be equal or weighted.

The training loss for unlabelled data may be any loss for unsupervised or semi-supervised training (e.g. uncertainty minimisation, self-reconstruction, normalized cut etc). The labelled and unlabelled data losses can either be treated equally or weighted.

A machine learning method may be distributed and multiple master student models and annotators/labellers may be combined across the distributed sites, and/or may combine their results.

Selection of annotated/labelled samples may be decided by a machine learning algorithm.

The data may comprise one or more of image data, text, audio or other structure data.

Annotation/labelling may be performed based on a consensus of several expert sources

Annotation/labelling may be crowd-sourced across a plurality of annotators/experts/labellers.

The master model may comprise an ensemble of trained models.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments are described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A data processing apparatus for training models on data, comprising processing circuitry configured to:
train a first model on a plurality of labelled data sets;
apply the first trained model to a plurality of non-labelled data sets to obtain first pseudo-labels;
train a second model using at least the labelled data sets, the non-labelled data sets and the first pseudo-labels;
apply the second trained model to non-labelled data sets to obtain second pseudo-labels; and
train a third model based on at least the labelled data sets, non-labelled data sets and the second pseudo-labels.

2. Apparatus according to claim 1, wherein the processing circuitry is configured to provide an output identifying data sets for labelling by a user and/or identifying at least some of the pseudo-labels for verification or modification by a user.

3. Apparatus according to claim 1, wherein the processing circuitry is configured to perform a series of training and labelling processes, thereby increasing the amount of the data that is labelled or pseudo-labelled and/or increasing an accuracy of the labelling and/or pseudo-labelling and/or increasing an accuracy of model output.

4. Apparatus according to claim 3, wherein the series of labelling processes comprise automatically pseudo-labelling data and/or labelling based on user input.

5. Apparatus according to claim 3, wherein the series of training and labelling processes comprises the training and applying of the first model, the training and applying of the second model, the training of the third model, an applying of the third model, and a training and applying of at least one further model such that N models are training and applied, where N is an integer.

6. Apparatus according to claim 5, wherein at least one of: N is greater than 2, N is greater than 3, N is between 3 and 20.

7. Apparatus according to claim 1, wherein the number of labelled data sets is at least one of: less than 50% of the number of said unlabelled data sets, less than 10% of the number of said unlabelled data sets, less than 1% of the number of said unlabelled data sets.

8. Apparatus according to claim 1, wherein at least one of:
a) the number of unlabelled data sets is at least one of: greater than 50, greater than 100, greater than 1000;
b) the number of labelled data sets is at least one of: greater than 1, between 1 and 1000, or between 1 and 100.

9. Apparatus according to claim 3, wherein the series of labelling and training processes is terminated in response to an output accuracy, a desired or predicted performance, an amount of labelled data, a number of iterations reaching a threshold value, or there being no or less than a threshold amount of improvement in comparison to a previous process in the series.

10. An apparatus according to claim 1, wherein the first, second and third models make up or form part of a series of models that are trained and applied, and the processing circuitry is configured to apply a final trained model of the series to a data set to obtain an output.

11. An apparatus according to claim 10, wherein the data set comprises a medical imaging data set and the output comprises or represent a classification and/or a segmentation and/or an identification of an anatomical feature or pathology.

12. An apparatus according to claim 1, wherein at least some of the training is based on a combination of classification and uncertainty minimisation.

13. An apparatus according to claim 12, wherein at least some of the training is based on determination of classification loss value(s) for the labelled data sets and determination of uncertainty minimisation loss value(s) for the unlabelled data sets and/or the labelled data sets alone or in combination.

14. An apparatus according to claim 12, wherein the uncertainty minimisation comprises estimating uncertainty using a dropout layer of one or more of the models.

15. An apparatus according to claim 1, wherein the processing circuitry is configured to determine a measure of uncertainty based on differences between predictions or other outputs of the models.

16. An apparatus according to claim 1, wherein the data comprises medical imaging data or text data.

17. An apparatus according to claim 1, wherein the data sets comprise at least one magnetic resonance (MR) data sets, computed tomography (CT) data sets, X-ray data sets, ultrasound data sets, positron emission tomography (PET) data sets, single photon emission computed tomography (SPECT) data sets, or patient record data sets.

18. An apparatus according to claim 1, wherein labels of the labelled sub-set(s) of data comprise or represent a classification and/or a segmentation and/or an identification of an anatomical feature or pathology.

19. A method of training models on data, comprising:
training a first model on a plurality of labelled data sets;
applying the first trained model to data plurality of non-labelled data sets to obtain first pseudo-labels;
training a second model using at least the labelled data sets, the non-labelled data sets and the first pseudo-labels;
applying the second trained model to non-labelled data sets to obtain second pseudo-labels; and
training a third model based on at least the labelled data sets, non-labelled data sets and the second pseudo-labels.

20. A method of processing data comprising applying a final model trained using an apparatus according to claim 10 to a data set thereby to obtain an output.
